(19) United States
(12) Patent Application Publication
(10) Pub. No.: US 2017/0035703 A1
(43) Pub. Date: Feb. 9, 2017

(54) Method of Treating Adhesion by Altering an Epithelial to Mesenchymal Transition

(71) Applicant: Hans M. Albertsen, Salt Lake City, UT (US)

(72) Inventor: Hans M. Albertsen, Salt Lake City, UT (US)

(21) Appl. No.: 15/296,175

(22) Filed: Oct. 18, 2016

Related U.S. Application Data
(60) Provisional application No. 62/247,847, filed on Oct. 29, 2015.

(51) Int. Cl. A61K 9/70; A61K 31/65; A61K 31/4412

(52) U.S. Cl. CPC A61K 9/7007; A61K 31/4412; A61K 31/65

(57) ABSTRACT

The present invention is a method of treating adhesion including the prevention or reduction of abdominal, pelvic, and thoracic adhesions, by administering an Epithelial-to-mesenchymal transition altering compound or device. The device may take the form of a substrate coated with an Epithelial-to-mesenchymal transition altering compound.

### CROSS REFERENCE TO RELATED APPLICATIONS

This US nonprovisional utility patent application claims the benefit under 35 USC §119(e) of U.S. provisional application No. 62/247,847 filed Oct. 29, 2015 which is incorporated herein in its entirety by this reference.

### FIELD OF THE INVENTION

The present invention relates to methods of treating and preventing adhesion, and more especially to methods of treating and preventing adhesion by altering an epithelial-to-mesenchymal transition (EMT).

### BACKGROUND OF THE INVENTION

Adhesions are the abnormal union of membranous surfaces of the human body and are often the result of inflammation or injury. Adhesions consist of fibrous bands and encapsulations of varying density and are commonly observed after surgical procedures (see Appx A). As used herein, Abdominal, Pelvic, and Thoracic Adhesion (APTA) shall be understood to be non-anatomical fibrous adhesions—specifically including adhesions that form following surgery, infection, inflammatory diseases, radiation, chemotherapy, and other trauma—that affect serosal surfaces of the abdominal and/or pelvic and/or thoracic cavity.

APTA is commonly associated with pain when adhesions between surfaces that normally move freely relative to one-another restricts free movement. Adhesions cause pain by pulling nerves, either within an organ tied down by an adhesion or within the adhesion itself. Other symptoms of APTA include nausea, vomiting, bloating, abdominal swelling, inability to have a bowel movement or pass gas, constipation and restricted breathing.

In addition to pain, other pathologies related to APTA include infertility and digestive tract obstructions.

APTA is a common post-surgical complication that depending on the method and purpose of surgery can occur in 40%-90% of surgical patients.

EMT is a process by which epithelial cells lose their cell polarity and cell-cell adhesion, and gain migratory and invasive properties to become mesenchymal stem cells (see Appx B). Mesenchymal stem cells are multipotent stromal cells that can differentiate into a variety of cell types (see Appx C). Stromal cells are connective tissue cells derived from mesenchyme of any organ, for example in the uterine mucosa (endometrium), prostate, bone marrow, and the ovary. They are cells that support the function of the parenchymal cells of the organ (see Appx D). EMT plays an important role in organ morphogenesis during embryonic development and in wound healing (Kalluri and Weinberg (2009), The basics of epithelial-mesenchymal transition. The Journal of Clinical Investigation. 119:1420-1428; Kim et al. (2014), Role of the epithelial-mesenchymal transition and its effects on embryonic stem cells. Experimental & Molecular Medicine. 46:e108; Lamouille et al. (2014), Molecular mechanisms of epithelial-mesenchymal transition. Nature Reviews Molecular Cell Biology. 15:178-196). EMT has been found to play a critical role in tumor invasion and metastasis (Loboda et al. (2011), EMT is the dominant program in human colon cancer. BMC Medical Genomics. 4:9), and in cancer stem-cell formation (Plaks et al. (2015), The cancer stem cell niche: how essential is the niche in regulating stemness of tumor cells? Cell Stem Cell. 16:225-238). Mesothelial-to-mesenchymal transition (MMT) refers to mesodermally derived epithelium (see Appx E). For the purposes of this application, unless indicated otherwise, EMT shall be considered to encompass MMT.

A method relating to treating endometrial tissue disease by altering an epithelial to mesenchymal transition is disclosed in U.S. patent applications 62/170,346 and Ser. No. 15/141,964 (published as US 2016/0250234 on Sep. 1, 2016) to Albertsen, which are expressly incorporated herein in their entirety by this reference.

### SUMMARY OF THE INVENTION

The present invention is a method of treating APTA by preventing or minimizing the development of an APTA by altering EMT in a patient at risk of developing an APTA. Exemplary patients at increased risk of having or developing APTA are patients undergoing surgery—especially abdominal or pelvic surgery—and patients having chronic disease. EMT of a patient is preferably altered by administering an EMT altering composition (EAC). The EAC specifically preferably affects cellular remodeling of epithelial membranes that prevents the development and/or progression an APTA. More specifically the treatment is intended to inhibit or reverse EMT—a prerequisite for developing APTA.

Depending on the degree of an APTA present in a patient, or the genetic and environmental risk to which a patient is exposed, a graded range of treatment is anticipated. Younger, asymptomatic patients may be treated by simple over-the-counter supplements to maintain good epithelial health and thus reduce the risk for developing APTA, whereas patients with significantly elevated risks may be treated by compositions available only by prescription. As all individuals have a unique genetic make-up and live in unique environments the EAC formulations may benefit from optimization of the formulations and dosages in a manner dependent on genetic and environmental factors specific to a given patient. Benefits of the disclosed EAC treatment method include for instance prevention of APTA, reduction in secondary APTA complications, reduction in APTA associated pain, and reduction in APTA associated infertility. Moreover, the treatment of an APTA asymptomatic patient with an EAC to prevent APTA onset or development wherein such APTA asymptomatic patient is found to have at least one APTA predisposing risk-factor like pelvic dialysis or an associated genetic marker (including for instance Single Nucleotide Polymorphisms or SNPs, Rare Variants or RVs, and Copy Number Variants or CNVs) in the DNA of the APTA asymptomatic patient is also contemplated. Similarly, patients exposed to potentially pathological microbial faunas, including vaginal microbiomes and sexually transmitted diseases encountered when having unprotected sex are also considered.

A set of markers characteristic of the epithelial state include E-cadherin (CDH1), whereas N-cadherin and Vimentin are characteristic of the mesenchymal state. EMT inhibitors include a range of known compounds that have been tested for efficacy in tissue-culture based experiments by measuring, for example, the ratio between E-cadherin and N-cadherin. Systems like these are being used to identify novel compounds for their ability to inhibit EMT and include for instance rapamycin, 17-AAG and LY294002, and A83-01 and GDC-0941 (Chua et al. (2012), A cell-based small molecule screening method for identifting inhibitors of epithelial-mesenchymal transition in carcinoma. PloS one. 7:e33183; Reka et al. (2011), Identifying inhibitors of epithelial-mesenchymal transition by connectivity map-based systems approach. Journal of Thoracic Oncology, official publication of the Internation Association for the study of Lung Cancer, 6:1784-1792). It is expected that the range of EAC compounds will grow and that members of this group will prove useful in the treatment of APTA via their effect on EMT.

### DESCRIPTION OF DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a trimetric view of a select EAC.

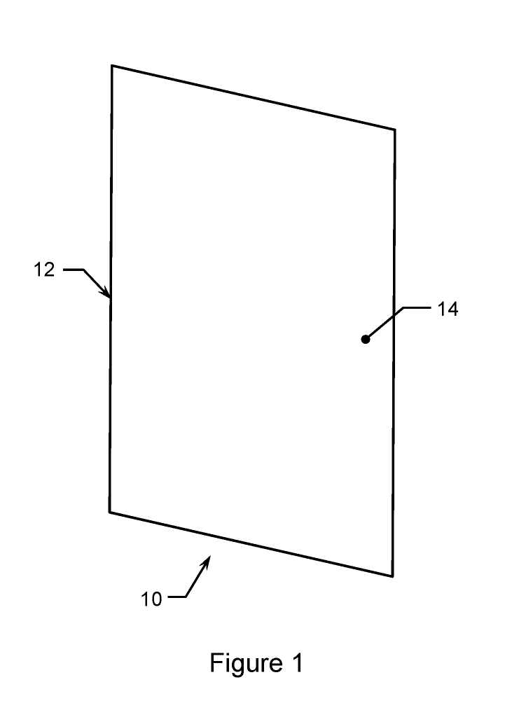

### DETAILED DESCRIPTION OF THE INVENTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are included to provide a thorough understanding of embodiments of the invention. One skilled in the relevant art will recognize, however, that the invention can be practiced without one or more of the specific details, or with other methods, components, materials, and so forth. In other instances, well-known structures, materials, or operations are not shown or described in detail to avoid obscuring aspects of the invention.

In order to facilitate the understanding of the present invention in reviewing the drawings accompanying the specification, a feature table is provided below. It is noted that like features are like numbered throughout all of the figures.

FEATURE TABLE

| # | Feature | # | Feature |
|---|---|---|---|
| 10 | Adhesion Prevention Sheet (APS) | 12 | Sheet substrate |
| 14 | EAC Coating | | |

The invention disclosed herein is a method of treating APTA by preventing occurrence of an APTA and/or minimizing the extent of APTA in a patient by administering to the patient an EAC in an amount effective to alter EMT in the patient. Included in such EMT alteration are EMT prevention, EMT inhibition, EMT reduction, and EMT reversal. Possible EACs include both natural and synthetic EACs. EACs may be specific microRNAs and protein-specific antibodies. Specific exemplary EACs include, Sorafenib, Metacycline, Diphenyl difluoroketone, Resveratrol, Curcumin, and Emodin (Dandawate et al. (2013), Novel strategies targeting cancer stem cells through phytochemicals and their analogs. Drug Delivery and Translational Research. 3:165-182; Diaz-Lopez et al. (2014), Role of microRNA in epithelial to mesenchymal transition and metastasis and clinical perspectives. Cancer Management and Research. 6:205-216; Hu et al. (2010), Curcumin inhibits transforming growth factor-beta activity via inhibition of Smad signaling in HK-2 cells. American Journal of Nephrology. 31:332-341; Ji et al. (2015), Resveratrol suppresses epithelial-to-mesenchymal transition in colorectal cancer through TGF-beta1/Smads signaling pathway mediated Snail/E-cadherin expression. BMC Cancer. 15:97; Jia et al. (2015), Sorafenib ameliorates renal fibrosis through inhibition of TGF-beta-induced epithelial-mesenchymal transition. PloS one. 10:e0117757; Li et al. (2013), Curcumin inhibits transforming growth factor-beta1-induced EMT via PPARgamma pathway, not Smad pathway in renal tubular epithelial cells. PloS one. 8:e58848; Li et al. (2013), Resveratrol inhibits the epithelial-mesenchymal transition of pancreatic cancer cells via suppression of the PI-3K/Akt/NF-kappaB pathway. Current Medicinal Chemistry. 20:4185-4194; Liu et al. (2015), miR-221 facilitates the TGFbeta1-induced epithelial-mesenchymal transition in human bladder cancer cells by targeting STMN1. BMC Urology. 15:36; Montgomery et al. (2014), MicroRNA mimicry blocks pulmonary fibrosis. EMBO Molecular Medicine. 6:1347-1356; Parvani et al. (2015), Silencing beta3 Integrin by Targeted ECO/siRNA Nanoparticles Inhibits EMT and Metastasis of Triple-Negative Breast Cancer. Cancer Research; Rico-Leo et al. (2013), Dioxin receptor expression inhibits basal and transforming growth factor beta-induced epithelial-to-mesenchymal transition. The Journal of Biological Chemistry. 288:7841-7856; Subramaniam et al. (2008), Diphenyl difluoroketone: a curcumin derivative with potent in vivo anticancer activity. Cancer Research. 68:1962-1969; Thacker and Karunagaran (2015), Curcumin and Emodin Down-Regulate TGF-beta Signaling Pathway in Human Cervical Cancer Cells. PloS one. 10:e0120045; Xi et al. (2014), Inhibition of epithelial-to-mesenchymal transition and pulmonary fibrosis by methacycline. American Journal of Respiratory Cell and Molecular Biology. 50:51-60; Zaravinos (2015), The Regulatory Role of MicroRNAs in EMT and Cancer. Journal of Oncology. 2015:865816; Zhang et al. (2015), Proton pump inhibitor pantoprazole abrogates adriamycin-resistant gastric cancer cell invasiveness via suppression of Akt/GSK-beta/beta-catenin signaling and epithelial-mesenchymal transition. Cancer Letters. 356:704-712; Zhang et al. (2015), MicroRNA-33b, upregulated by EF24, a curcumin analog, suppresses the epithelial-to-mesenchymal transition (EMT) and migratory potential of melanoma cells by targeting HMGA2. Toxicology Letters. 234:151-161). Methods of administration of such EACs include oral consumption, intravenous, subcutaneous or intra-muscular injection, intra-abdominal injection, rectal absorption, and trans-vaginal deposition. In the case of a patient manifesting symptoms of an APTA, the EAC may be for instance administered to the patient without further analysis of the patient. In the case of an APTA asymptomatic patient, the EAC may be for instance administered to the patient after having determined that the patient possess at least one APTA associated biomarker, such as a specific protein or peptide, or a specific inherited DNA variant known to be associated with increased risk for developing APTA.

EACs are envisioned to be administered either as a single compound or as a combination of multiple EACs. An EAC may for instance be administered in combination with a nonsteroidal anti-inflammatory drug (NSAID) or in combination with a hormonal treatment such as an oral contraceptive (OC).

In a first exemplary embodiment of the invention, the invention comprises the step of administering to a patient known to have an APTA or to manifest symptoms of an APTA, an oral administration of an EAC of Sorafenib in pill form, resulting in the reversal of EMT in the patient and resulting in the reduction or reduced progression of APTA symptoms in the patient. Alternatively, the EAC may be in the form of a topical gel or cream or further alternatively, the EAC may be in the form of spray (e.g. so as to spray onto the intended site of treatment).

In a second exemplary embodiment of the invention, the invention comprises the step of administering to a patient being asymptomatic of an APTA but known to have an acute risk of APTA (e.g. scheduled abdominal surgery), an intra-venous injection of an EAC of Metacycline in liquid form, resulting in the inhibition of EMT in the patient and resulting in prevention of APTA symptoms in the patient.

Referring now to the drawing, a third exemplary embodiment of the invention is an Adhesion Prevention Sheet (APS) 10, comprising a substantially planar and preferably flexible and drapable sheet substrate 12 having a therapeutically effective EAC coating 14 adhered thereon. Sheet substrate 12 preferable is constructed of a material that is biologically compatible to human implantation and preferably dissolves over time when placed in contact with bodily fluids. Moreover, at least a portion of a surface of sheet substrate 12 is coated with EAC coating 14, but preferable both (major) sides of sheet substrate 12 are substantially completely coated with EAC coating 14.

In a fourth exemplary embodiment of the invention, the invention comprises the steps of performing a surgical operation on a patient and applying APS 10 to a patient such that APS 10 is positioned adjacent to a first serosal surface of the abdominal or pelvic cavity but preferably in between a first serosal surface of the abdominal or pelvic cavity and a second serosal surface of the abdominal or pelvic cavity such that an adhesion of such serosal surface of the abdominal or pelvic cavity is prevented. Thus for instance, APS 10 could be positioned between an abdominal wall surface and an organ wall surface or alternatively APS 10 could be positioned between a first organ wall surface and a second organ wall surface. In practice, APS 10 dissolves over time, EMT is altered, an APTA is prevented, and the pain and symptoms of an APTA are avoided or reduced.

In additional to reducing or preventing the symptoms of APTA, a benefit of the disclosed invention is that the disclosed treatment of APTA avoids follow-up surgery to remove adhesions.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method of treating adhesion in a subject comprising administering an EAC to said subject such that EMT of said subject is altered.

2. The method of claim 1, wherein said adhesion defines an APTA.

3. The method of claim 1, wherein said EAC defines an APS.

4. The method of claim 1, wherein said EAC defines at least one of a natural EAC and a synthetic EAC.

5. The method of claim 1, wherein said EAC defines at least one of Sorafenib, Metacycline, Diphenyl difluoroketone, Resveratrol, Curcumin, and Emodin.

6. The method of claim 1, wherein said EAC defines an EAC in at least one of a pill form, a liquid form, a cream form, a spray form, and a suppository form.

7. The method of claim 1, wherein said administering defines at least one of an oral administration, an intravenous administration, a subcutaneous administration, an intra-muscular injection administration, an intra-abdominal injection administration, a rectal absorption administration, a trans-vaginal deposition administration, and a surgical implantation.

8. The method of claim 1, wherein said subject is adhesion asymptomatic.

9. The method of claim 3, wherein the EAC coating of said APS includes at least one of Sorafenib, Metacycline, Diphenyl difluoroketone, Resveratrol, Curcumin, and Emodin.

10. The method of claim 1, wherein at least one of the abdominal cavity, the pelvic cavity, and the thoracic cavity of said subject has experienced at least one of inflammation, injury, and surgery.

11. The method of claim 1, wherein said method includes the step of surgically implanting an APS in proximity to a serosal surface of at least one of the abdominal cavity, the pelvic cavity, and the thoracic cavity of said subject.

12. The method of claim 11, wherein said APS is positioned between a first serosal surface and a second serosal surface such that an adhesion of said serosal surfaces is prevented.

13. An adhesion prevention device comprising a generally planar substrate having at least a portion of at least one surface thereof coated with an EAC composition.

14. The device of claim 13, wherein said substrate defines a substantially flexible, pliable, and drapable substrate.

15. The device of claim 13, wherein said substrate defines a human biocompatible substrate.

16. The device of claim 13, wherein said substrate defines a dissolvable substrate that dissolves when exposed to human bodily fluids for at least a predetermined duration of time.

17. The device of claim 13, wherein said EAC composition includes at least one of Sorafenib, Metacycline, Diphenyl difluoroketone, Resveratrol, Curcumin, and Emodin.

18. The device of claim 13, wherein a majority of the external surface of said substrate is coated with said EAC composition.

19. The device of claim 13, wherein said device is positioned within at least one of an abdominal cavity, a pelvic cavity, and a thoracic cavity.

20. The device of claim 19, wherein said device is positioned between a first serosal surface and a second serosal surface such that a first surface of said device is positioned in surface contact to said a first serosal surface and a second surface of said device is positioned in surface contact to a second serosal surface such that an adhesion of said serosal surfaces is prevented.
